(19) United States
(12) Patent Application Publication
(10) Pub. No.: US 2025/0090720 A1
(43) Pub. Date: Mar. 20, 2025

(54) TUBULAR SCAFFOLD AND METHOD OF MANUFACTURING THE SAME

(71) Applicants: THE CATHOLIC UNIVERSITY OF KOREA INDUSTRY-ACADAMIC COOPERATION FOUNDATION, Seoul (KR); GACHON UNIVERSITY OF INDUSTRY-ACADEMIC COOPERATION FOUNDATION, Gyeonggi-do (KR)

(72) Inventors: Sung Won KIM, Seoul (KR); Sun Hwa PARK, Seoul (KR); Jin Woo LEE, Incheon (KR); Young Soo YU, Incheon (KR)

(21) Appl. No.: 18/891,155

(22) Filed: Sep. 20, 2024

(30) Foreign Application Priority Data
Sep. 20, 2023 (KR) 10-2023-0125375

(51) Int. Cl. A61L 27/18; A61F 2/00; A61F 2/04; A61F 2/06; A61L 27/36; A61L 27/38; D01D 5/00

(52) U.S. Cl. CPC A61L 27/18; A61F 2/06; A61L 27/3691; A61L 27/3817; A61L 27/3882; D01D 5/003; A61F 2002/0081; A61F 2002/044; A61F 2002/046; A61F 2210/0076; A61F 2230/0069; A61F 2240/001; A61L 2400/12; A61L 2430/22; D10B 2331/041; D10B 2509/00

(57) ABSTRACT

The present invention relates to a tubular scaffold and a method of manufacturing the same. The tubular scaffold has physical properties matching the characteristics of a tubular organ that bends or rotates when a double layer is formed by electrospinning nanofibers, and not only has excellent cell adhesion ability but may also maintain cell functions. Also, since such effects have been proven in an animal model, the tubular scaffold may be mass-produced and used as a highly functional tubular organ having various sizes and lengths.

### CROSS-REFERENCE TO RELATED APPLICATION

This application claims priority to and the benefit of Korean Patent Application No. 10-2023-0125375, filed on Sep. 20, 2023, the disclosure of which is incorporated herein by reference in its entirety.

### BACKGROUND

### 1. Field of the Invention

The present invention relates to a tubular scaffold and a method of manufacturing the same.

### 2. Discussion of Related Art

Regenerative medicine involves repairing, regenerating, maintaining, and replacing tissues and organs using exogenous materials such as scaffolds. Target tissues or organs may be artificially produced by seeding cells such as primary cells, stem cells, or the like and various factors for tissue growth on these scaffolds.

A scaffold is an artificial extracellular matrix (ECM) artificially manufactured for tissue construction and cell function control, and basically must have biocompatible properties so as not to induce transplant rejection, cytotoxicity, inflammatory responses, and the like. Therefore, scaffolds suitable for specific tissues or organs require various characteristics, that is, they must reproduce the specific characteristics of each tissue or organ, and have high efficiency in cell injection and cell proliferation induction.

Meanwhile, fibrous scaffolds produced by electrospinning have attracted much attention as a way to manufacture a scaffold because they can mimic the structure of natural ECM. In particular, electrospun scaffolds may be manufactured using various materials such as polymers, proteins, metals, ceramics, and the like, and are known to have a very high surface-to-volume ratio and have the cell patterning function obtained by aligning fibers in a certain direction.

For these reasons, electrospun scaffolds have been used to regenerate tissues such as blood vessels, bones, peripheral and central nervous systems, and the like. However, despite these advantages, the electrospun scaffolds are not widely used as a tool for tissue regeneration in actual clinical practice. The reason for this is investigated that the design and characteristics of the 3D pattern that serves as the basic foundation of the scaffold, and further, the characteristics of the targeted tissues or organs are not reflected.

Against this technical background, there is a need to develop scaffolds suitable for regenerative medicine and tissue engineering and suitable for target tissues or organs, but no significant research results have been reported yet.

### RELATED ART DOCUMENT

### Patent Document

Korean Patent Publication No. 10-2023-0114228

### SUMMARY OF THE INVENTION

Accordingly, it is an object of the present invention to provide a double-layered tubular scaffold, wherein the scaffold is characterized by being nanofiberized.

It is another object of the present invention to provide a method of manufacturing a double-layered tubular scaffold, which includes a nanofiberizing step.

It is still another object of the present invention to provide a kit for manufacturing a double-layered tubular scaffold, which includes a composition for nanofiberization; and instructions.

It is yet another object of the present invention to provide a method of fiberizing a double-layered tubular scaffold, which includes electronspinning nanofibers, wherein the electronspinning is characterized by the following conditions: the density of polycaprolactone (PCL) ranges from 0.1 to 50 (w/v %);
the solvent is any one selected from chloroformic acid or formic acid; and
the voltage ranges from 5 to 50 kV.

However, the technical objects to be achieved by the present invention are not limited to the above-described technical objects, and other objects which are not mentioned above will be clearly understood from the following detailed description by those skilled in the art to which the present invention pertains.

According to an aspect of the present invention, there is provided a double-layered tubular scaffold, wherein the scaffold is characterized by being nanofiberized.

According to one embodiment of the present invention, the double layer may be composed of an inner layer and an outer layer, and the diameter of the outer layer may be larger than that of the inner layer, but the present invention is not limited thereto.

According to one embodiment of the present invention, the diameter ratio of the inner layer and the outer layer may range from 1:3 to 9, but the present invention is not limited thereto.

According to one embodiment of the present invention, the outer layer of the tubular scaffold may have grooves having a size of 0.1 mm to 50 mm, but the present invention is not limited thereto.

According to one embodiment of the present invention, the tubular scaffold may be any one or more selected from the group consisting of a blood vessel, a trachea, and an esophagus, but the present invention is not limited thereto.

According to one embodiment of the present invention, the tubular scaffold may not include a hollow space, but the present invention is not limited thereto.

According to another aspect of the present invention, there is provided a method of manufacturing a double-layered tubular scaffold, which includes a nanofiberizing step.

According to one embodiment of the present invention, the nanofiberizing step may be performed by applying any one method selected from an electrospinning method or a culturing method, but the present invention is not limited thereto.

According to one embodiment of the present invention, the electrospinning method may be a method in which the density of polycaprolactone (PCL) ranges from 0.1 to 50 (w/v %), but the present invention is not limited thereto.

According to one embodiment of the present invention, the electrospinning method may be a method in which a solvent is any one selected from the group consisting of chloroformic acid, formic acid, acetic acid, acetone, dichloromethane, 1,2-dichloroethane, dimethylformamide, ethanol, isopropyl alcohol, water, and tetrahydrofuran, but the present invention is not limited thereto.

According to one embodiment of the present invention, the electrospinning method may be a method in which a voltage ranges from 5 to 50 kV, but the present invention is not limited thereto.

According to still another aspect of the present invention, there is provided a kit for manufacturing a double-layered tubular scaffold, which includes a composition for nanofiberization; and instructions.

According to one embodiment of the present invention, the instructions may include the above contents, but the present invention is not limited thereto.

According to yet another aspect of the present invention, there is provided a method of nanofiberizing a double-layered tubular scaffold, which includes: electronspinning nanofibers,
wherein the electronspinning is characterized by the following conditions:
the density of polycaprolactone (PCL) ranges from 0.1 to 50 (w/v %);
the solvent is any one selected from chloroformic acid or formic acid; and
the voltage ranges from 5 to 50 kV.

According to yet another aspect of the present invention, there is provided a use of the double-layered tubular scaffold for the manufacture of a tubular organ.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a drawing showing the appearance of a nano-sized trachea scaffold (trachea scaffold+E) manufactured according to the method of the present invention;

FIG. 2A shows the results of SEM analysis of the trachea scaffold+E manufactured according to the method of the present invention, and FIG. 2B is an image comparing the trachea scaffold+E with a non-nanofiberized trachea scaffold (trachea scaffold);

FIGS. 3A and 3B are images showing the appearance of the trachea scaffold+E manufactured according to the method of the present invention and the trachea scaffold before and after a torsion test, respectively;

FIGS. 3C and 3D are graphs showing the rotation angle and its average value, respectively, confirmed in the torsion test on the trachea scaffold+E manufactured according to the method of the present invention and the trachea scaffold;

FIG. 4 is an image showing the results of analyzing the attachment ability of the trachea scaffold+E manufactured according to the method of the present invention to cells;

FIGS. 5A and 5B show the results of analysis of the survival rates of chondrocytes and chondrocyte differentiation markers in the trachea scaffold+E manufactured according to the method of the present invention and the trachea scaffold, respectively; and

FIGS. 6A and 6B are images showing the results of transplanting the trachea scaffold+E manufactured according to the method of the present invention and the trachea scaffold into an animal model, respectively.

### DETAILED DESCRIPTION OF EXEMPLARY EMBODIMENTS

The present invention provides a double-layered tubular scaffold, wherein the scaffold is characterized by being nanofiberized.

In the present invention, “scaffold” may include anything that may become a structure that may replace a part of a damaged organ or tissue in the living body and supplement or replace their functions. Also, when the scaffold is used in vivo or ex vivo as one of the artificial organs or tissues, a scaffold to which specific cells are attached is used. In the related art, the scaffold may be used interchangeably with the term “cell support” and the like, but the present invention is not limited thereto.

In the present invention, the number, diameter, and thickness of layers formed by the nanofibers spun onto the scaffold, and the ratio of the thickness of each layer may affect cell proliferation and attachment and a cell survival rate. In this case, the cell survival rate may mean whether, when the organ scaffold on which the nanofiber layers are formed is transplanted into an animal model, the cells may attach to the organ scaffold and maintain their actual cell function while surviving at a high level like an organ in vivo.

According to one embodiment of the present invention, the double layer may be composed of an inner layer and an outer layer, and the diameter of the outer layer may be larger than that of the inner layer, but the present invention is not limited thereto.

According to one embodiment of the present invention, the diameter ratio of the inner layer and the outer layer may range from 1:3 to 9, but the present invention is not limited thereto.

In the present invention, the diameter ratio of the inner layer and the outer layer may range from 1:3 to 9, 1:3 to 8, 1:3 to 7, 1:3 to 6, 1:3 to 5.9, 1:3 to 5.8, 1:3 to 5.71, 1:4 to 9, 1:4 to 8, 1:4 to 7, 1:4 to 6, 1:4 to 5.9, 1:4 to 5.8, 1:4 to 5.71, 1:5 to 9, 1:5 to 8, 1:5 to 7, 1:5 to 6, 1:5 to 5.9, 1:5 to 5.8, 1:5 to 5.71, 1:5.2 to 9, 1:5.2 to 8, 1:5.2 to 7, 1:5.2 to 6, 1:5.2 to 5.9, 1:5.2 to 5.8, 1:5.2 to 5.71, 1:5.4 to 9, 1:5.4 to 8, 1:5.4 to 7, 1:5.4 to 6, 1:5.4 to 5.9, 1:5.4 to 5.8, 1:5.4 to 5.71, 1:5.6 to 9, 1:5.6 to 8, 1:5.6 to 7, 1:5.6 to 6, 1:5.6 to 5.9, 1:5.6 to 5.8, 1:5.6 to 5.71, 1:5.7 to 9, 1:5.7 to 8, 1:5.7 to 7, 1:5.7 to 6, 1:5.7 to 5.9, 1:5.7 to 5.8, or 1:5.7 to 5.71, or may preferably range from 1:5.71, which may mean a ratio of 0.7:4, but the present invention is not limited thereto.

According to one embodiment of the present invention, the outer layer of the tubular scaffold may have grooves having a size of 0.1 mm to 50 mm, but the present invention is not limited thereto.

In the present invention, the outer layer of the tubular scaffold may include grooves having a size of 0.1 mm to 30 mm, 0.1 mm to 10 mm, 0.1 mm to 5 mm, 0.1 mm to 2 mm, 0.5 mm to 30 mm, 0.5 mm to 10 mm, 0.5 mm to 5 mm, 0.5 mm to 2 mm, 1 mm to 30 mm, 1 mm to 10 mm, 1 mm to 5 mm, 1 mm to 2 mm, 1.3 mm to 30 mm, 1.3 mm to 10 mm, 1.3 mm to 5 mm, 1.3 mm to 4 mm, 1.3 mm to 3 mm, 1.3 mm to 2 mm, 1.5 mm to 30 mm, 1.5 mm to 10 mm, 1.5 mm to 5 mm, 1.5 mm to 4 mm, 1.5 mm to 3 mm, 1.5 mm to 2 mm, 1.8 mm to 30 mm, 1.8 mm to 10 mm, 1.8 mm to 5 mm, 1.8 mm to 4 mm, 1.8 mm to 3 mm, or 1.8 mm to 2 mm, or preferably a size of 2 mm, but the present invention is not limited thereto.

In the present invention, the outer layer of the tubular scaffold may include grooves having a certain thickness. These grooves are generated through electrospinning of nanofibers and thus have a distinguishing feature from the plate-like 3D pattern used in conventional artificial tubular scaffolds. Accordingly, when the tubular scaffold is treated with chondrocytes, the effects of increasing the survival rate and maintaining functions such as cell differentiation ability may occur, but the present invention is not limited thereto.

In the present invention, the scaffold may be in a tubular shape, and may be a tubular tissue or organ. According to one embodiment of the present invention, the tubular scaffold may be any one or more selected from the group consisting of a blood vessel, a trachea, and an esophagus, but the present invention is not limited thereto.

In general, tubular cells, tissues or organs such as blood vessels or organs are characterized by having a hollow space, and structures such as stents that are inserted into blood vessels of the human body to keep the inside of the vessel open are mainly formed to have a circular hollow space. However, according to one embodiment of the present invention, the tubular scaffold may not include a hollow space, but the present invention is not limited thereto.

In general, tubular cells are required to have flexibility, such as lateral bending, longitudinal elongation, or the like, and the tubular tissues or organs are also required to have an ability to withstand forces such as rotation, bending, and twisting, and have flexible characteristics, and the like. The tubular scaffold of the present invention may have excellent physical properties required for tubular cells, tissues, or organs because the nanofibers are formed into a double layer and all of the hollow spaces of the 3D pattern applied to the tubular scaffold are covered, but the present invention is not limited thereto.

The present invention provides a method of manufacturing a double-layered tubular scaffold, which includes a nanofiberizing step.

In the present invention, the term “nanofiberizing step” may refer to a process of blocking the hollow space of a tubular organ by treating nanofibers on a cell support such as a 3D pattern for manufacturing a tubular scaffold, but the present invention is not limited thereto.

To the “nanofiberizing step” of the present invention, any method capable of nanofiberization may be applied, and may mean the widest range of nanofiberization methods, such as floating culture capable of nanofiberization through culturing a 3D pattern. According to one embodiment of the present invention, any one method selected from an electrospinning method or a culturing method may be applied to the nanofiberizing step, but the present invention is not limited thereto.

In the present invention, the term “electrospinning” refers to a method of manufacturing nanofibers by forming an electric field between a tip from which a polymer solution or melt is spun and a collector that accommodates nanofibers. Depending on the direction between the collector and the tip, the electrospinning may be divided into upward, downward, and horizontal electrospinning. In general, upward or downward electrospinning is widely applied.

Factors affecting the electrospinning may include material factors, process factors, and environmental factors. The material factors include the chemical structure, molecular weight, polymer solution concentration, viscosity, surface tension, permittivity, salt concentration, and the like of the polymer constituting an electrospinning solution, the process factors include electrospinning conditions such as the voltage, temperature of a solution or melt, the inner and outer diameters, spinning distance and angle of the spinneret tip, the movement, speed, directionality, and the like of a dust collection unit such as a plate type, a drum type, a conveyor belt type, and the like, and the environmental factors include the temperature, humidity, air current, and the like of the atmospheric environment during spinning. In particular, when electrospinning is performed on a structure to be used in the living body, completely different types of nanofiberization may proceed depending on the conditions such as the type of solvents such as a solvent, PCL, and the like, the density of solvents, the spraying distance, the thickness of nanofibers, the number of nanofiber layers, voltage, and the like.

According to one embodiment of the present invention, the electrospinning method may be a method in which the density of polycaprolactone (PCL) ranges from 0.1 to 50 (w/v %), but the present invention is not limited thereto.

In the present invention, the PCL density conditions may be applied differently depending on the inner or outer layer of the scaffold.

In the present invention, for the inner layer of the scaffold, the PCL density may range from 0.1 to 50 (w/v %), 0.1 to 40 (w/v %), 0.1 to 30 (w/v %), 0.1 to 25 (w/v %), 0.1 to 20 (w/v %), 1 to 50 (w/v %), 1 to 40 (w/v %), 1 to 30 (w/v %), 1 to 25 (w/v %), 1 to 20 (w/v %), 10 to 50 (w/v %), 10 to 40 (w/v %), 10 to 30 (w/v %), 10 to 25 (w/v %), 10 to 20 (w/v %), 13 to 50 (w/v %), 13 to 40 (w/v %), 13 to 30 (w/v %), 13 to 25 (w/v %), 13 to 20 (w/v %), 16 to 50 (w/v %), 16 to 40 (w/v %), 16 to 30 (w/v %), 16 to 25 (w/v %), 16 to 20 (w/v %), 18 to 50 (w/v %), 18 to 40 (w/v %), 18 to 30 (w/v %), 18 to 25 (w/v %), or 18 to 20 (w/v %), or may be 20 (w/v %), but the present invention is not limited thereto.

In the present invention, for the outer layer of the scaffold, the PCL density may range from 0.1 to 50 (w/v %), 0.1 to 40 (w/v %), 0.1 to 30 (w/v %), 0.1 to 20 (w/v %), 0.1 to 15 (w/v %), 1 to 50 (w/v %), 1 to 40 (w/v %), 1 to 30 (w/v %), 1 to 20 (w/v %), 1 to 15 (w/v %), 10 to 50 (w/v %), 10 to 40 (w/v %), 10 to 30 (w/v %), 10 to 20 (w/v %), 10 to 15 (w/v %), 12 to 50 (w/v %), 12 to 40 (w/v %), 12 to 30 (w/v %), 12 to 20 (w/v %), 12 to 15 (w/v %), 13 to 50 (w/v %), 13 to 40 (w/v %), 13 to 30 (w/v %), 13 to 20 (w/v %), 13 to 15 (w/v %), 14 to 50 (w/v %), 14 to 40 (w/v %), 14 to 30 (w/v %), 14 to 20 (w/v %), or 14 to 15 (w/v %), or may be 15 (w/v %), but the present invention is not limited thereto.

According to one embodiment of the present invention, the electrospinning method may be a method in which the solvent is any one selected from the group consisting of chloroformic acid, formic acid, acetic acid, acetone, dichloromethane, 1,2-dichloroethane, dimethylformamide, ethanol, isopropyl alcohol, water, and tetrahydrofuran, but the present invention is not limited thereto.

In the present invention, the solvent used in the electrospinning method may be different depending on the inner layer or the outer layer. Preferably, formic acid may be applied to the inner layer and chloroformic acid may be applied to the outer layer, but the present invention is not limited thereto.

According to one embodiment of the present invention, the electrospinning method may be a method in which the voltage ranges from 5 to 50 kV, but the present invention is not limited thereto.

In the present invention, the voltage conditions for the electrospinning method may be different depending on the inner layer or the outer layer.

In the present invention, the voltage applied to the inner layer may range from 5 to 50 kV, 5 to 40 kV, 5 to 30 kV, 5 to 25 kV, 10 to 50 kV, 10 to 40 kV, 10 to 30 kV, 10 to 25 kV, 15 to 50 kV, 15 to 40 kV, 15 to 30 kV, 15 to 25 kV, 20 to 50 kV, 20 to 40 kV, 20 to 30 kV, 20 to 28 kV, 20 to 26 kV, 20 to 25 kV, 22 to 50 kV, 22 to 40 kV, 22 to 30 kV, 22 to 28 kV, 22 to 26 kV, 22 to 25 kV, 23 to 50 kV, 23 to 40 kV, 23 to 30 kV, 23 to 28 kV, 23 to 26 kV, 23 to 25 kV, 24 to 50 kV, 24 to 40 kV, 24 to 30 kV, 24 to 28 kV, 24 to 26 kV, or 24 to 25 kV, or may be 25 kV, but the present invention is not limited thereto.

In the present invention, the voltage applied to the outer layer may range from 5 to 50 kV, 5 to 40 kV, 5 to 30 kV, 5 to 26.5 kV, 10 to 50 kV, 10 to 40 kV, 10 to 30 kV, 10 to 26.5 kV, 15 to 50 kV, 15 to 40 kV, 15 to 30 kV, 15 to 26.5 kV, 20 to 50 kV, 20 to 40 kV, 20 to 30 kV, 20 to 28 kV, 20 to 26.5 kV, 22 to 50 kV, 22 to 40 kV, 22 to 30 kV, 22 to 28 kV, 22 to 26.5 kV, 23 to 50 kV, 23 to 40 kV, 23 to 30 kV, 23 to 28 kV, 23 to 26.5 kV, 24 to 50 kV, 24 to 40 kV, 24 to 30 kV, 24 to 28 kV, 24 to 26.5 kV, 25 to 50 kV, 25 to 40 kV, 25 to 30 kV, 25 to 28 kV, 25 to 26.5 kV, 26 to 50 kV, 26 to 40 kV, 26 to 30 kV, 26 to 28 kV, or 26 to 26.5 kV, or may be 26.5 kV, but the present invention is not limited thereto.

The present invention provides a kit for manufacturing a double-layered tubular scaffold, which includes a composition for nanofiberization; and instructions.

According to one embodiment of the present invention, the instructions may include the above contents, but the present invention is not limited thereto.

In the present invention, the term “kit” refers to a tool for manufacturing a double-layered tubular scaffold by performing nanofiberization on a 3D pattern, which includes a preparation such as a composition for nanofiberization. In addition to the preparation, the kit of the present invention may include other components, compositions, solutions, devices, and the like that are usually necessary for a method of measuring or detecting the same. Specific examples may further include a 3D pattern for a tubular scaffold to be nanofiberized, components necessary for the storage and management of the composition for nanofiberization, and the like, but the present invention is not limited thereto. In this case, each component may be applied at least once without any limitation in the number of times, and there is no limitation in the order in which each material is applied. In this case, the application of each material may be performed simultaneously or randomly.

In the present invention, the kit may further include a container; instructions; or the like. The container may serve to package the preparation, and may also serve to store and fix the preparation. The material of the container may take the form of, for example, a bottle, a tub, a sachet, an envelope, a tube, an ampoule, and the like, which may be formed partly or wholly from plastics, glass, paper, foil, wax, and the like. The container may be initially equipped with a completely or partially detachable stopper, which is part of the container or which may be attached to the container by mechanical, adhesive, or other means. Also, the container may also be equipped with a stopper through which the contents may be accessed by a syringe needle. The kit may include an outer package, and the outer package may include instructions regarding the use of the components.

The present invention provides a method of nanofiberizing a double-layered tubular scaffold, which includes:
electronspinning nanofibers,
wherein the electronspinning is characterized by the following conditions:
the density of polycaprolactone (PCL) ranges from 0.1 to 50 (w/v %);
the solvent is any one selected from chloroformic acid or formic acid; and
the voltage ranges from 5 to 50 kV.

Hereinafter, preferred examples of the present invention are presented in order to aid in understanding the present invention. However, it should be understood that the following examples are provided only to make the present invention easier to understand and are not intended to limit the present invention.

### EXAMPLES

### Example 1: Manufacture of Nano-Sized Trachea Scaffold as Tubular Scaffold

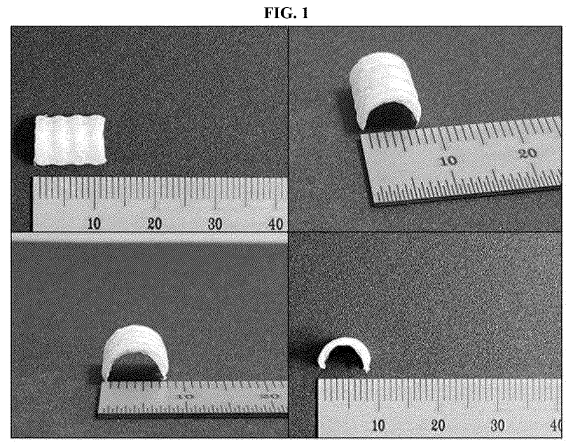

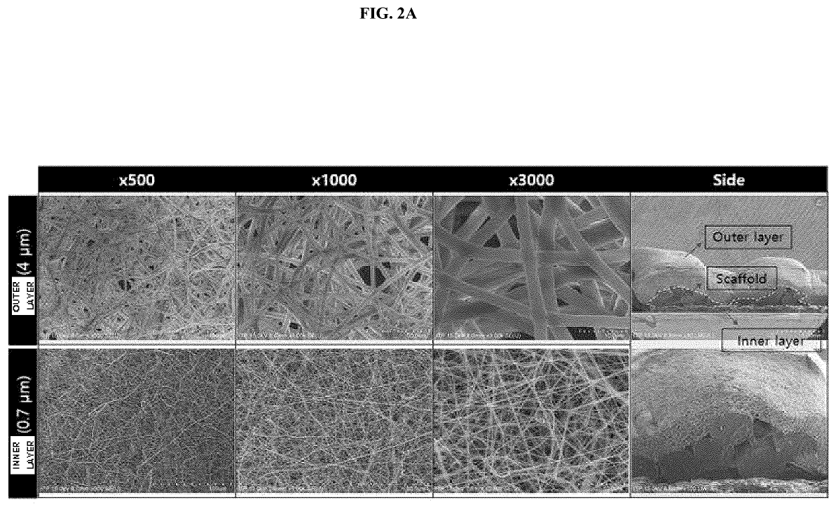

A nano-sized trachea scaffold (trachea scaffold+E) was manufactured as the tubular scaffold of the present invention. Specifically, a double-layered tubular trachea scaffold was manufactured by spinning nanofibers onto a 3D pattern (randomly-oriented pattern) under the conditions of Table 1 using an electrospinning machine (Nano Nc—ESR200RD) (FIG. 1).

TABLE 1

| | Conditions | | | |
|---|---|---|---|---|
| | Fiber diameter | PCL density | | Voltage |
| Classification | (μm) | (w/v %) | Solvent | (kV) |
| Inner layer | 0.7 | 20 | Formic acid | 25 |
| Outer layer | 4 | 15 | Chloroform | 26.5 |

### Example 2: Characterization of Trachea Scaffold+E

The characteristics of the trachea scaffold+E manufactured in Example 1 were confirmed. Specifically, the trachea was observed at 500, 1,000, and 3,000 magnifications using a scanning electron microscope (SEM), and the lateral structure was confirmed.

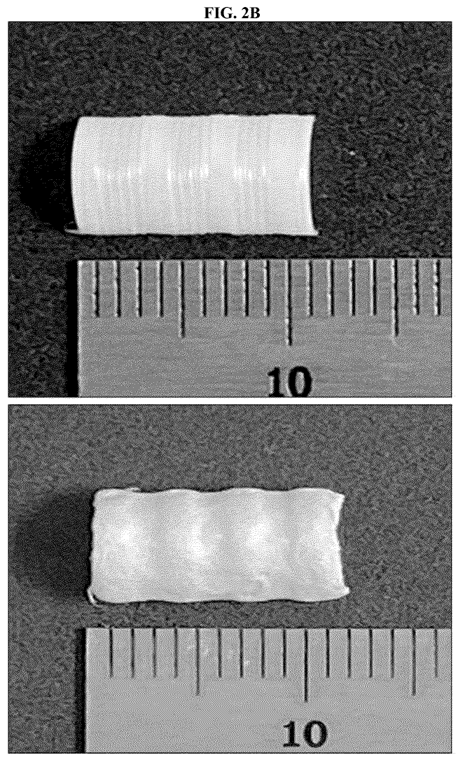

As a result, the trachea scaffold+E manufactured in Example 1 was manufactured with a thickness of 400 μm. Also, it was confirmed that a film of irregularly overlapping nanofibers was formed on the 3D pattern as the nanofibers were electrospun, and thus the hollow space of the 3D pattern was completely covered (FIG. 2A). In addition, it was confirmed that the plate-shaped 3D pattern had grooves having a size of approximately 2 mm through the electrospinning of the nanofibers (FIG. 2B).

### Example 3: Confirmation of Excellent Physical Properties of Trachea Scaffold+E

Basically, since the neck including the trachea is able to bend and rotate, the neck has an essential ability to withstand forces such as bending and rotation in order to stably maintain the functions of the lumen and structure after the artificial trachea is transplanted. Therefore, a torsion test was performed to analyze the physical properties of the trachea scaffold+ manufactured in Example 1. Specifically, the trachea scaffold (Frame only), which is the 3D pattern used in Example 1 and on which the nanofibers were not spun, was used as the control for the trachea scaffold+E (Frame+E) of Example 1. After both sides of the trachea scaffold+E and the trachea scaffold were fixed to a fixed support, 10° torsion was applied to observe the appearance of each trachea, and the rotation angle was measured. Each test was performed in triplicate.

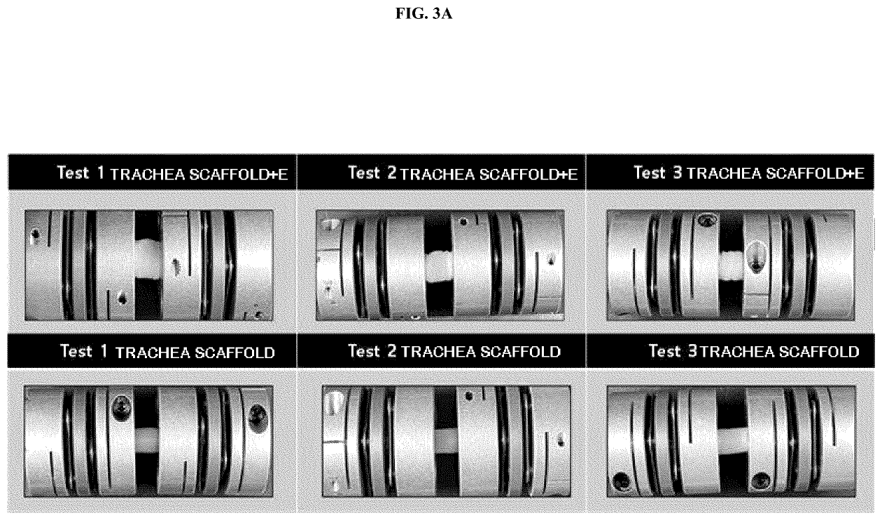

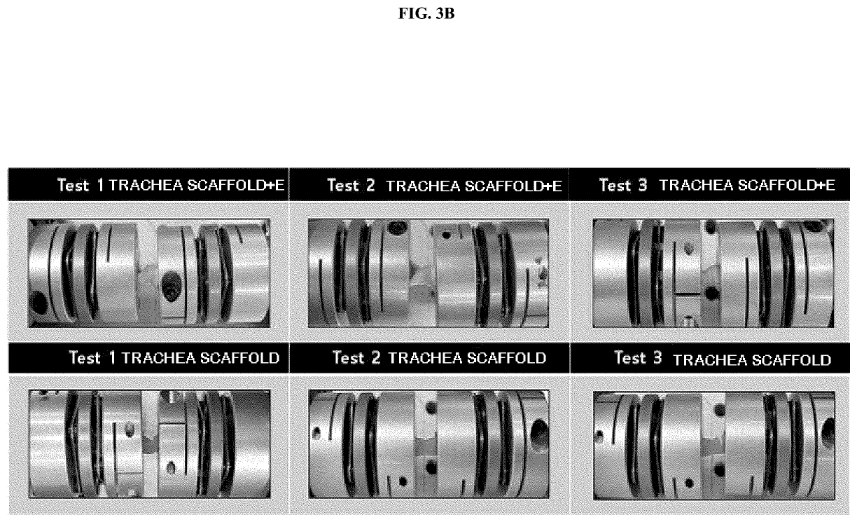

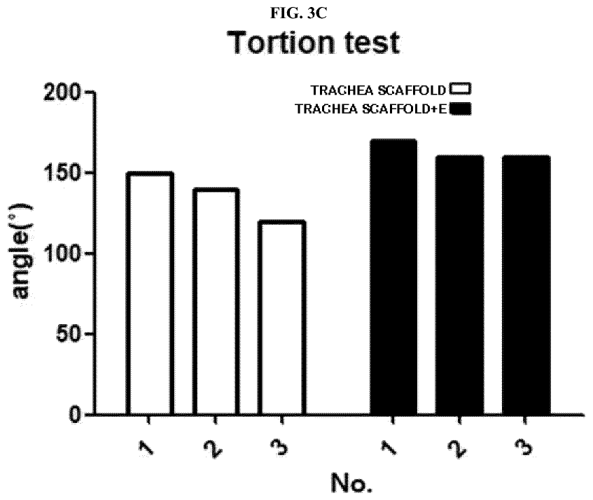

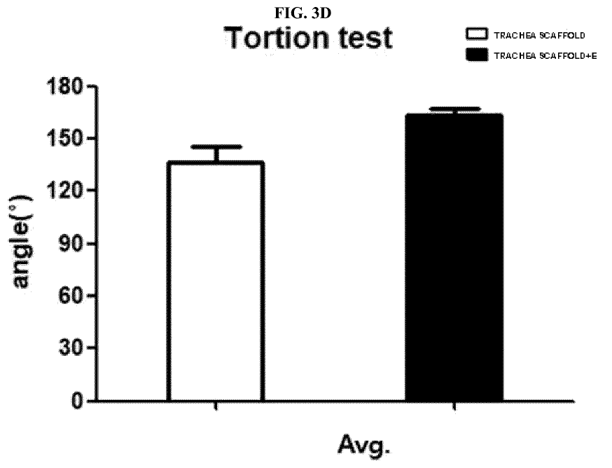

As a result, the appearance of the trachea scaffold+E and the trachea scaffold before and after the torsion test are shown in FIGS. 3A and 3B, confirming that the trachea scaffold+E of the present invention has physical properties suitable for bending, rotation, and tensile forces.

Also, the rotation angles of the trachea scaffold+E and the trachea scaffold were confirmed as in Table 2 and FIG. 3C, and the average of the rotation angles of each trachea scaffold was confirmed as in FIG. 3D. Based on these results, it was confirmed that the trachea scaffold+E of Example 1 had a larger rotation angle than the trachea scaffold in all three experiments (Table 2 and FIG. 3C). Also, it was confirmed that the average rotation angle of the trachea scaffold+E was approximately 163.3°, while the rotation angle of the trachea scaffold approximately 136.6°. Accordingly, the rotation angle of the trachea scaffold+E was found to correspond to 120% of the trachea scaffold (FIG. 3D).

TABLE 2
Rotation angle (°) of torsion test

| Classification | Test 1 | Test 2 | Test 3 |
|---|---|---|---|
| Trachea scaffold + E | 170 | 160 | 160 |
| Trachea scaffold | 150 | 140 | 120 |

That is, it was confirmed that the trachea scaffold+E of the present invention has the characteristics of maintaining the function of an actual trachea as is in vitro, in vivo, and in animal and clinical tests in that the flexibility as well as the rotational motion ability were significantly enhanced because the hollow spaces of the 3D pattern were all covered by the nanofiber spinning.

### Example 4: Confirmation of Excellent Cell Adhesion Ability of Trachea Scaffold+E

The cell adhesion ability of the trachea scaffold+E manufactured in Example 1 was analyzed. Specifically, human nasal septal chondrocyte cells (hNCs) were seeded on the trachea scaffold+E manufactured in Example 1, and cultured for 24 hours to observe the degree of cell adhesion.

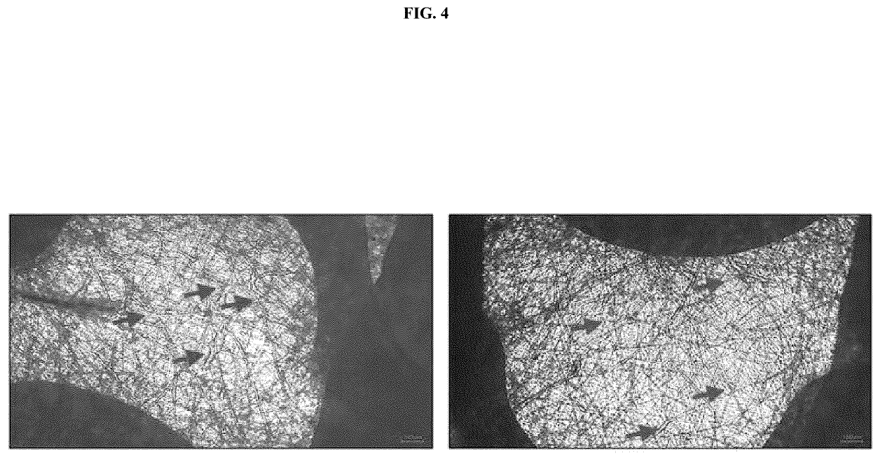

As a result, it was confirmed that chondrocytes were attached to the trachea scaffold+E at an excellent level depending on the shape of the nanofibers (FIG. 4).

### Example 5: Confirmation of Excellent Chondrocyte Survival Rate and Characteristics of Trachea Scaffold+E

In Example 4, the excellent adhesion ability was confirmed when chondrocytes were sprayed onto the trachea scaffold+E of the present invention. The survival rate and characteristics of chondrocytes attached to the trachea scaffold+E due to such high adhesion ability were analyzed.

### Example 5-1: Analysis of Survival Rate of Chondrocytes

The cell adhesion ability of the trachea scaffold+E according to Example 1 was analyzed using a Live/Dead kit. Specifically, the same number of hNCs as in Example 4 were seeded on the trachea scaffold+E of the present invention under the same conditions, and cultured for 7 days. Thereafter, fluorescence staining was performed using the Live/Dead kit according to the instructions, and observed at 200 μm. In this case, the same non-nanfiberized trachea scaffold as in Example 3 was used as the control.

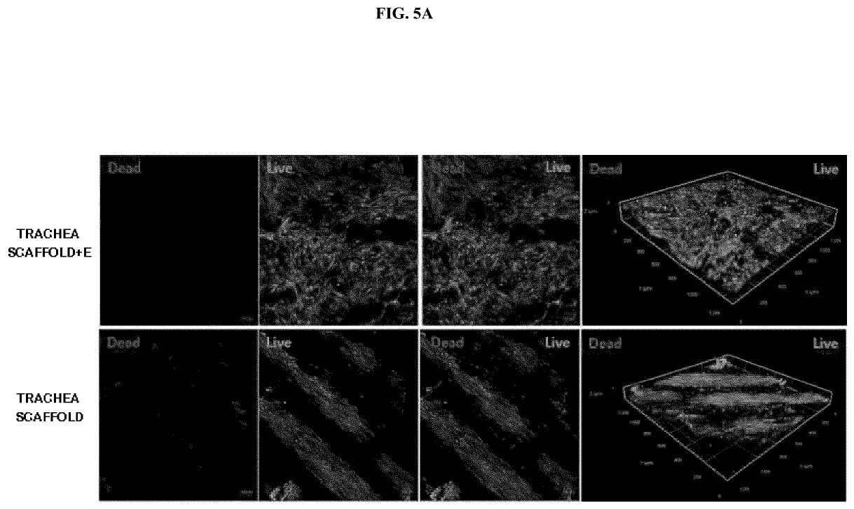

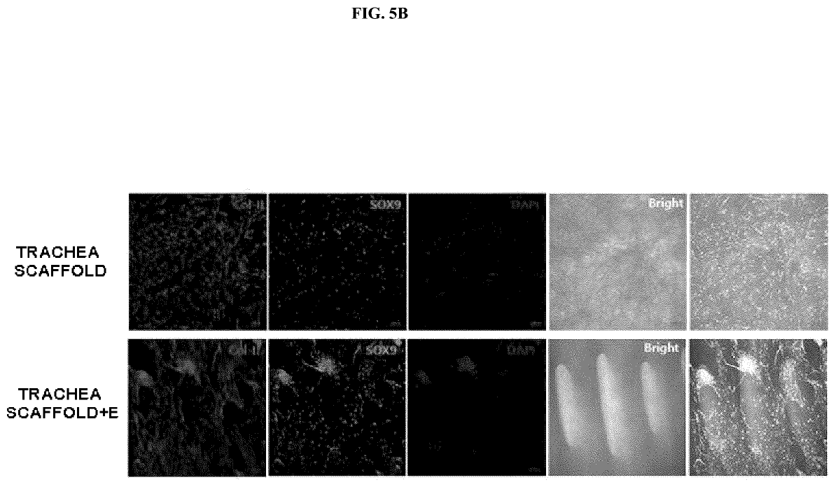

As a result, in the trachea scaffold as the control, although the chondrocytes were attached along the 3D pattern, dead cells were observed in the curved parts. On the other hand, it was confirmed that the chondrocytes in the trachea scaffold+E of the present invention were attached in a fiber shape, indicating that the living cells were evenly distributed. This result suggests that the attached chondrocytes may maintain a high cell survival rate when the chondrocytes are evenly distributed and attached (FIG. 5A).

### Example 5-2: Characterization of Chondrocytes

The characteristics of chondrocytes were analyzed in the same manner as in Example 5-1. Specifically, sox9 and col-II were identified as markers for analyzing the characteristics of chondrocytes.

As a result, it was confirmed that the characteristics of the attached chondrocytes were well maintained (FIG. 5B).

### Example 6: Confirmation of Excellent In Vivo Functionality of Trachea Scaffold+E

To confirm the in vivo functionality of the trachea scaffold+E manufactured in Example 1, the trachea scaffold+E was transplanted into a rabbit animal model. Specifically, the trachea scaffold+E of Example 1 was transplanted into a rabbit animal model, and endoscopic observation and autopsy were performed two months later. In this case, the same non-nanfiberized trachea scaffold as in Example 3 was used as the control.

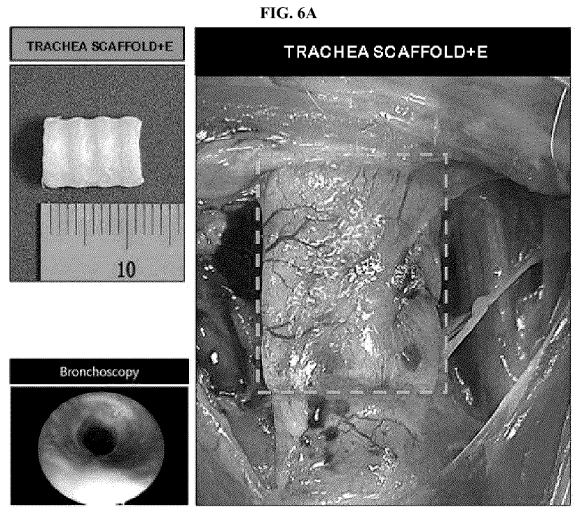

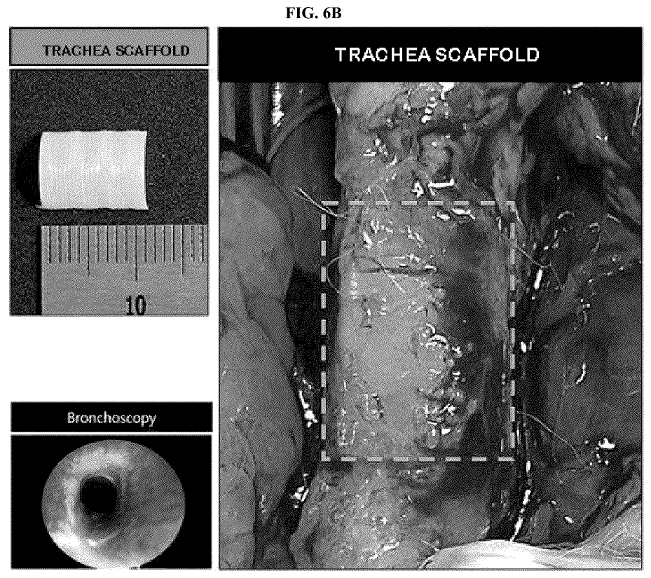

Based on the results (FIGS. 6A and 6B), it was confirmed that a significantly larger amount of microvessels were generated when the trachea scaffold+E of Example 1 was transplanted, even though trachea scaffolds having the same size were transplanted. Also, it was confirmed that the morphology of connective tissue was observed around the mucosa and cartilage, and the trachea scaffold+E was effective in maintaining the lumen (FIG. 6A). On the other hand, it was confirmed that the non-nanfiberized trachea scaffold was effective in maintaining the lumen, but had a lower level of generation of microvessels compared to the test group (FIG. 6B).

According to the tubular scaffold and the method of manufacturing the same, the tubular scaffold has physical properties matching the characteristics of a tubular organ that bends or rotates when a double layer is formed by electrospinning nanofibers, and not only has excellent cell adhesion ability but can also maintain cell functions. Also, since such effects have been proven in an animal model, the tubular scaffold can be mass-produced and used as a tubular organ having various sizes and lengths.

The description of the present invention described above is for illustrative purposes, and it should be understood that those of ordinary skill in the art to which the present invention pertains can easily modify embodiments into other specific forms without changing the technical idea or essential features described in this specification. Therefore, it should be understood that all the embodiments described above are illustrative in all respects and not restrictive.

1. A double-layered tubular scaffold, wherein the scaffold is nanofiberized.

2. The tubular scaffold of claim 1, wherein the double layer is composed of an inner layer and an outer layer, and
the diameter of the outer layer is larger than that of the inner layer.

3. The tubular scaffold of claim 2, wherein the diameter ratio of the inner layer and the outer layer ranges from 1:3 to 9.

4. The tubular scaffold of claim 2, wherein the outer layer of the tubular scaffold has grooves having a size of 0.1 mm to 50 mm.

5. The tubular scaffold of claim 1, wherein the tubular scaffold is any one or more selected from the group consisting of a blood vessel, a trachea, and an esophagus.

6. The tubular scaffold of claim 1, wherein the tubular scaffold does not include a hollow space.

7. The tubular scaffold of claim 1, wherein the tubular scaffold is manufactured by a method for manufacturing a double-layered tubular scaffold, comprising a nanofiberizing step.

8. The tubular scaffold of claim 7, wherein the nanofiberizing step is performed by applying any one method selected from an electrospinning method or a culturing method.

9. The tubular scaffold of claim 8, wherein the electrospinning method is a method in which the density of polycaprolactone (PCL) ranges from 0.1 to 50 (w/v %).

10. The tubular scaffold of claim 8, wherein the electrospinning method is a method in which a solvent is any one selected from the group consisting of chloroformic acid, formic acid, acetic acid, acetone, dichloromethane, 1,2-dichloroethane, dimethylformamide, ethanol, isopropyl alcohol, water, and tetrahydrofuran.

11. The tubular scaffold of claim 8, wherein the electrospinning method is a method in which a voltage ranges from 5 to 50 kV.

12. (canceled)

13. (canceled)

14. (canceled)

15. A method of manufacturing a double-layered tubular scaffold, comprising a nanofiberizing step.

16. The method of claim 15, wherein the nanofiberizing step is performed by applying any one method selected from an electrospinning method or a culturing method.

17. The method of claim 16, wherein the electrospinning method is a method in which the density of polycaprolactone (PCL) ranges from 0.1 to 50 (w/v %).

18. The method of claim 16, wherein the electrospinning method is a method in which a solvent is any one selected from the group consisting of chloroformic acid, formic acid, acetic acid, acetone, dichloromethane, 1,2-dichloroethane, dimethylformamide, ethanol, isopropyl alcohol, water, and tetrahydrofuran.

19. The method of claim 16, wherein the electrospinning method is a method in which a voltage ranges from 5 to 50 kV.
